(19) United States
(12) Patent Application Publication
(10) Pub. No.: US 2022/0354761 A1
(43) Pub. Date: Nov. 10, 2022

(54) COMPOSITIONS AND METHODS FOR TREATING KERATIN FIBERS

(71) Applicant: L'Oreal, Paris (FR)

(72) Inventors: Cho-Cho KHINE, Bridgewater, NJ (US); Ronak RUGHANI, Edison, NJ (US)

(21) Appl. No.: 17/733,920

(22) Filed: Apr. 29, 2022

Related U.S. Application Data
(60) Provisional application No. 63/182,263, filed on Apr. 30, 2021.

(30) Foreign Application Priority Data
Jul. 22, 2021 (FR) 2107919

(51) Int. Cl. A61K 8/362; A61K 8/365; A61K 8/73; A61Q 5/04

(52) U.S. Cl. CPC A61K 8/362; A61K 8/365; A61K 8/738; A61Q 5/04

(57) ABSTRACT

The disclosure relates to compositions comprising (a) at least one carboxylic acid or salt thereof; (b) at least one cyclodextrin or derivative thereof; and (c) at least one solvent. The compositions can be used for providing lasting curl definition and/or frizz and/or volume control to hair, even in high humidity. The disclosure also relates to methods of making and using the compositions.

### TECHNICAL FIELD

The present disclosure relates to compositions and methods for treating keratin fibers, such as curly or wavy hair. The compositions improve the definition of hair curls and/or provide frizz and/or volume control to the hair. The disclosure also relates to methods of making and using the compositions.

### BACKGROUND

Consumers with curly or wavy hair are often dissatisfied with the appearance of their hair. For example, curly hair may appear frizzy, with poor curl definition. Although products exist that are meant to reduce frizziness and/or improve curl definition, these products have drawbacks such as imparting a greasy feel to the hair, weighing the hair down, or being ineffective, particularly in humid climates. As such, there is a desire for new and improved products that can enhance the visual appearance of curliness of the hair, such as by improving the definition of the curls, and/or reducing the volume and/or frizziness of the hair.

It has now surprisingly been found that compositions comprising at least one carboxylic acid and at least one cyclodextrin are able to improve the appearance of curly or wavy hair, for example by improving curl definition and curl hold, and/or providing frizz control. These benefits have been shown to persist, even in conditions of high humidity.

### SUMMARY

The present disclosure relates to compositions for treating keratin fibers, such as curly or wavy hair, by improving curl definition and/or frizz control, and/or reducing the visual volume of the hair, as well as methods of using the compositions.

In one embodiment, the disclosure relates to compositions comprising (a) at least one carboxylic acid or salt thereof, (b) at least one cyclodextrin or derivative thereof; and (c) at least one solvent. In various embodiments, the compositions have a mole ratio of (a):(b) ranging from about 0.5:1 to about 25:3, such as about 4:1 to about 15:1 or about 6:1 to about 10:1.

In various embodiments, the (a) at least one carboxylic acid or salt thereof may be chosen from mono-carboxylic acids, di-carboxylic acids, tri-carboxylic acids, salts thereof, or mixtures thereof, such as, for example citric acid, lactic acid, malic acid, maleic acid, tartaric acid, glycolic acid, salts thereof, or mixtures thereof. In further embodiments, a polycarboxylic acid having more than three carboxyl group, such as phytic acid, may be used, alone or in combination with one or more mono-carboxylic acids, di-carboxylic acids, tri-carboxylic acids, salts thereof, or mixtures thereof. The (a) at least one carboxylic acid or salt thereof may, in various embodiments, be present in an amount ranging from about 0.01% to about 20% by weight, relative to the weight of the composition, such as, for example, about 0.1% to about 10% or about 0.5% to about 8%.

In various embodiments, the (b) at least one cyclodextrin or derivative thereof may be chosen from compounds of formula:

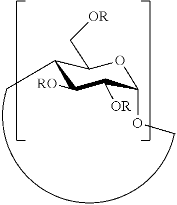

where R is chosen from H, CH₃, or a hydroxypropyl group, and n ranges from 6 to 8. For example, the at least one cyclodextrin or derivative thereof may be chosen from α-cyclodextrin, β-cyclodextrin, γ-cyclodextrin, methyl-α-cyclodextrin, methyl-β-cyclodextrin, methyl-γ-cyclodextrin, or mixtures thereof. The (b) at least one cyclodextrin or derivative thereof may, in various embodiments, be present in an amount ranging from about 0.01% to about 20% by weight, relative to the weight of the composition, such as, for example, about 0.1% to about 10% or about 0.4% to about 6%.

The disclosure further relates to methods of using the compositions, for example methods of treating hair by applying the compositions according to the disclosure to the hair. The methods may impart one or more of improved curl definition and/or volume control and/or frizz control to the hair, which may in various embodiments last for one or several hours even at high humidity.

The disclosure further relates to methods of making the compositions, wherein by choosing specific weight or mole ratios of the components, compositions providing particular degrees of curl definition and/or volume control and/or frizz control can be attained.

### BRIEF DESCRIPTION OF THE FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 is a graph demonstrating the curl definition provided to hair by compositions C1-C4 and 1A at T0 and T24H.

FIGS. 2A-2E are images of swatches of hair treated with compositions C1-C4 and 1A taken at T0, T0_{comb}, and T24H.

FIGS. 3A-3B are graphs demonstrating the effective frizz (3A) and effective frizz area (3B) of hair treated with compositions C1-C4 and 1A at T0, T1H, and T24H.

FIGS. 4A-4E are images of swatches of hair treated with compositions C1-C4 and 1A taken at T0, T0.5H, T1H, T2H, and T24H.

FIGS. 5A-5B are graphs demonstrating the effective frizz (5A) and effective frizz area (5B) of hair treated with compositions C1 and 2A-2B at T0, T1H, and T24H.

FIGS. 6A-6C are images of swatches of hair treated with compositions C1 and 2A-2B taken at T0 and T24H.

FIGS. 7A-7B are images of swatches of hair treated with compositions C1 and 2C-2D taken at T0 and T1H.

FIGS. 8A-8B are images of swatches of hair treated with compositions 2E-2G taken at T0 and T1H.

FIGS. 9A-9B are images of swatches of hair treated with compositions 3A-3D taken at T0 and T1H, with a comparative swatch treated with composition C1 at T1H.

FIGS. 10A-10C are graphs demonstrating the frizz (10A), curl definition (10B), and curliness (10C) of hair treated with compositions C1 and 4A-4F at T4H.

FIGS. 11A-11B are graphs demonstrating the frizz (11A) and frizz area (11B) of hair treated with compositions C1 and 4A-4F at T4H.

FIGS. 12A-12G are images of swatches of hair treated with compositions C1 and 4A-4F at T4H.

It is to be understood that the foregoing and following descriptions are exemplary and explanatory only, and are not intended to be restrictive of any subject matter claimed.

### DETAILED DESCRIPTION

The disclosure relates to compositions for styling hair, such as for improving the appearance of curly hair, which in various embodiments may provide one or more of curl definition, curl hold, and/or frizz control, as well as methods of making and using the compositions.

### I. Compositions

In various embodiments, compositions according to the disclosure comprise (a) at least one carboxylic acid; (b) at least one cyclodextrin; and (c) at least one solvent.

### Carboxylic Acid

Compositions according to the disclosure comprise at least one carboxylic acid, which includes salts thereof. The carboxylic acids which may be used include, in various embodiments, mono-carboxylic acids (one carboxylic group) or poly-carboxylic acids (two or more carboxylic groups). Preferably, the carboxylic acids are non-polymeric carboxylic acids. The carboxylic acids may be chosen, in certain embodiments, from those having a molecular weight of less than about 900 g/mol, such as less than about 700 g/mol, less than about 500 g/mol, less than about 300 g/mol, or less than about 200 g/mol.

In some embodiments, the at least one carboxylic acid may be chosen from mono-carboxylic acids such as formic acid, acetic acid, propionic acid, butyric acid, valeric acid, caproic acid, entanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, lactic acid, or salts thereof.

In further embodiments, the at least one carboxylic acid may be chosen from di-carboxylic acids such as oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, or salts thereof.

In yet further embodiments, the at least one carboxylic acid may be chosen from tri-carboxylic acids such as citric acid, isocitric acid, aconitric acid, propane-1,2,3-tricarboxylic acid, benzene-1,3,5-tricarboxylic acid, or salts thereof.

In still further embodiments, the at least one carboxylic acid may be chosen from poly-carboxylic acids that have more than three carboxylic groups, such as phytic acid.

In various embodiments, the compositions comprise a mixture of two or more carboxylic acids or salts thereof. For example, the compositions may comprise a mixture of two or more mono-carboxylic acids or salts thereof, or may comprise a mixture or two or more poly-carboxylic acids or salts thereof, such as two or more di-carboxylic acids, two or more tri-carboxylic acids, or a mixture of one or more di-carboxylic acids and one or more tri-carboxylic acids. Mixtures of one or more mono-carboxylic or salts thereof with one or more poly-carboxylic acids or salts thereof are also contemplated. In further embodiments, the composition comprises only one carboxylic acid or a salt thereof.

In certain embodiments, the at least one carboxylic acid is chosen from citric acid, lactic acid, malic acid, maleic acid, tartaric acid, glycolic acid, phytic acid, salts thereof, or a mixture of two or more of the foregoing.

In various embodiments, the compositions comprise a total amount of carboxylic acids and/or salt thereof ranging from about 0.01% to about 20% by weight, such as about 0.1% to about 18%, about 0.5% to about 15%, or about 1% to about 13% by weight, relative to the total weight of the composition. By way of non-limiting example, the compositions may comprise a total amount of carboxylic acids and/or salt thereof ranging from about 0.5% to about 15%, by weight, for example, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 15%, from about 2% to about 12%, from about 2% to about 10%, from about 2% to about 9%, from about 2% to about 8%, from about 2% to about 7%, from about 2% to about 6%, or from about 2% to about 5%, by weight, relative to the total weight of the composition, including all ranges and subranges thereof. In some embodiments, the compositions comprise a total amount of carboxylic acids and/or salt thereof ranging from about 0.5% to about 13%, preferably from about 0.5% to about 10%, and more preferably from about 0.5% to about 8% by weight, relative to the total weight of the composition.

In one embodiment, the at least one carboxylic acid comprises or consists of citric acid, lactic acid, malic acid, maleic acid, tartaric acid, glycolic acid, phytic acid, salts thereof, or a mixture thereof, and is present in an amount ranging from about 0.1% to about 10%, such as 0.3% to about 9%, or about 0.5% to about 8% by weight, relative to the total weight of the composition, including all ranges and subranges thereof.

### Cyclodextrin

Compositions according to the disclosure comprise at least one cyclodextrin, which includes derivatives thereof. Cyclodextrins are a family of cyclic oligosaccharides consisting of a macrocyclic ring of glucose subunits joined by α-1,4 glycosidic bonds.

The cyclodextrins that can be used include those of the formula:

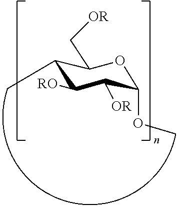

wherein:
   R is chosen from H, CH₃, or a hydroxypropyl group, and n ranges from 6-8.

For example, in embodiments where R=H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R=CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-B-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodexctrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

In various embodiments, the compositions comprise a total amount of cyclodextrins ranging from about 0.01% to about 20% by weight, such as about 0.1% to about 18%, about 0.5% to about 15%, or about 1% to about 13% by weight, relative to the total weight of the composition. By way of non-limiting example, the compositions comprise a total amount of cyclodextrins ranging from about 0.5% to about 15%, by weight, for example, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 15%, from about 2% to about 12%, from about 2% to about 10%, from about 2% to about 9%, from about 2% to about 8%, from about 2% to about 7%, from about 2% to about 6%, or from about 2% to about 5% by weight, relative to the total weight of the composition, including all ranges and subranges thereof. In some embodiments, the compositions comprise a total amount of cyclodextrins ranging from about 1% to about 13%, preferably from about 1% to about 7%, and more preferably from about 1% to about 5% by weight, relative to the total weight of the composition.

In one embodiment, the at least one cyclodextrin comprises, consists essentially of, or consists of 3-cyclodextrin, and is present in an amount ranging from about 0.1% to about 10%, such as 0.2% to about 8%, about 0.3% to about 7%, or about 0.4% to about 6% by weight, relative to the total weight of the composition, including all ranges and subranges thereof.

### Solvent

Compositions according to the disclosure comprise at least one solvent. In various embodiments, the solvent comprises, consists essentially of, or consists of water. In a preferred embodiment, the solvent consists essentially of water and the total amount of water ranges from about 50% to 99.9%, based on the total weight of the composition.

Compositions according to the disclosure may optionally comprise a cosmetically acceptable solvent other than water, for example in combination with water. By way of non-limiting example, the solvent other than water may be chosen from monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples include ethylene glycol, propylene glycol, butylene glycol, hexylene glycol, propanediol, and glycerin. Mixtures of the above can also be chosen, including mixtures of the above with water.

The total amount of solvent in the composition may range from about 50% to about 99.9% by weight, relative to the total weight of the composition, including all ranges and subranges therebetween. For example, the total amount of solvent may range from about 70% to about 99%, about 75% to about 99%, about 80% to 99%, about 85% to 99%, about 90% to about 99%, about 95% to about 99%, about 97% to about 99%, about 70% to about 98%, about 75% to about 98%, about 80% to 98%, about 85% to 98%, about 90% to about 98%, about 95% to about 98%, about 70% to about 95%, about 75% to about 95%, about 80% to 95%, about 85% to 95%, about 90% to about 95%, about 70% to about 90%, about 75% to about 90%, about 80% to 90%, or about 85% to 90% by weight, relative to the total weight of the composition, including all ranges and subranges thereof.

### Additional Components

Compositions according to the disclosure may optionally comprise any additional or auxiliary component suitable for use in such compositions. Such components may include, but are not limited to, thickening agents, conditioning agents, dyes/pigments, moisturizing agents, fillers, structuring agents, shine agents, antioxidants, fragrances, buffers, dispersants, plant extracts, preserving agents (preservatives), opacifiers, sunscreen agents, vitamins, pH adjusting agents, and antistatic agents.

Optional additional or auxiliary components may be present in an amount ranging up to about 15%, such as from about 0.01% to about 15%, about 0.01% to about 10% by weight, about 0.01% to about 5% by weight, or about 0.01% to about 1% by weight, relative to the total weight of the composition.

In various embodiments, the compositions may have a pH less than or equal to about 7. For example, the pH of the compositions may range from about 2 to about 6, such as from about 3 to about 5, about 3.5 to about 5, about 3 to about 4.5, about 3.5 to about 4.5, or from about 3 to about 4, including all ranges and subranges therebetween.

Compositions according to the disclosure are typically in the form of a liquid, cream, or lotion, but are not so limited. In some embodiments, the compositions are a non-aerosol type product, which may be dispensed from a bottle, tube, or spray-bottle. When the compositions are formulated as aerosol type products, the compositions may further comprise at least one propellant.

### II. Methods

### A. Methods of Making the Compositions

The disclosure also relates to methods of making compositions described herein. It may, in some embodiments, be particularly beneficial to choose specific mole or weight ratios of carboxylic acid(s), cyclodextrin(s), and/or solvent(s) (e.g. water). For example, by choosing specific mole or weight ratios of these components, it is possible to tailor the compositions so that greater or lesser degrees of curl definition and/or frizz control may be imparted.

Therefore, it may be useful to prepare a pre-mixture of carboxylic acid(s), cyclodextrin(s), and solvent(s), which may then be diluted to the desired concentration with additional solvent(s) and other additives before the composition is applied to the hair. By way of example, the pre-mixture may be prepared having a weight ratio of carboxylic acid(s):cyclodextrin(s):solvent(s) ranging from about 0.3:1:0.2 to about 5:1:0.3. For example, the weight ratio may be about 2:3:1, about 1:1:3, about 5:3:1, about 8:3:1, or about 10:3:1. The pre-mixture may, in various embodiments, have a mole ratio of carboxylic acid(s):cyclodextrin(s):solvent(s) ranging from about 0.5:1:5 to about 25:3:90. For example, the mole ratio may be about 1:1:10, about 2:1:10, about 3:1:10, about 4:1:10, about 5:1:10, about 6:1:10, about 7:1:10, about 8:1:10, about 9:1:10, about 10:1:10, about 11:1:10, about 12:1:10, about 13:1:10, about 14:1:10, about 15:1:10, about 16:1:10, about 17:1:10, about 18:1:10, about 19:1:10, about 20:1:10, about 1:1:15, about 2:1:15, about 3:1:15, about 4:1:15, about 5:1:15, about 6:1:15, about 7:1:15, about 8:1:15, about 9:1:15, about 10:1:15, about 11:1:15, about 12:1:15, about 13:1:15, about 14:1:15, about 15:1:15, about 16:1:15, about 17:1:15, about 18:1:15, about 19:1:15, about 20:1:15, about 1:1:20, about 2:1:20, about 3:1:20, about 4:1:20, about 5:1:20, about 6:1:20, about 7:1:20, about 8:1:20, about 9:1:20, about 10:1:20, about 11:1:20, about 12:1:20, about 13:1:20, about 14:1:20, about 15:1:20, about 16:1:20, about 17:1:20, about 18:1:20, about 19:1:20, about 20:1:20, about 1:1:25, about 2:1:25, about 3:1:25, about 4:1:25, about 5:1:25, about 6:1:25, about 7:1:25, about 8:1:25, about 9:1:25, about 10:1:25, about 11:1:25, about 12:1:25, about 13:1:25, about 14:1:25, about 15:1:25, about 16:1:25, about 17:1:25, about 18:1:25, about 19:1:25, about 20:1:25, about 1:1:30, about 2:1:30, about 3:1:30, about 4:1:30, about 5:1:30, about 6:1:30, about 7:1:30, about 8:1:30, about 9:1:30, about 10:1:30, about 11:1:30, about 12:1:30, about 13:1:30, about 14:1:30, about 15:1:30, about 16:1:30, about 17:1:30, about 18:1:30, about 19:1:30, about 20:1:30, about 1:1:35, about 2:1:35, about 3:1:35, about 4:1:35, about 5:1:35, about 6:1:35, about 7:1:35, about 8:1:35, about 9:1:35, about 10:1:35, about 11:1:35, about 12:1:35, about 13:1:35, about 14:1:35, about 15:1:35, about 16:1:35, about 17:1:35, about 18:1:35, about 19:1:35, about 20:1:35, about 1:1:40, about 2:1:40, about 3:1:40, about 4:1:40, about 5:1:40, about 6:1:40, about 7:1:40, about 8:1:40, about 9:1:40, about 10:1:40, about 11:1:40, about 12:1:40, about 13:1:40, about 14:1:40, about 15:1:40, about 16:1:40, about 17:1:40, about 18:1:40, about 19:1:40, about 20:1:40, about 1:1:45, about 2:1:45, about 3:1:45, about 4:1:45, about 5:1:45, about 6:1:45, about 7:1:45, about 8:1:45, about 9:1:45, about 10:1:45, about 11:1:45, about 12:1:45, about 13:1:45, about 14:1:45, about 15:1:45, about 16:1:45, about 17:1:45, about 18:1:45, about 19:1:45, about 20:1:45, about 1:1:50, about 2:1:50, about 3:1:50, about 4:1:50, about 5:1:50, about 6:1:50, about 7:1:50, about 8:1:50, about 9:1:50, about 10:1:50, about 11:1:50, about 12:1:50, about 13:1:50, about 14:1:50, about 15:1:50, about 16:1:50, about 17:1:50, about 18:1:50, about 19:1:50, about 20:1:50, about 1:2:10, about 2:2:10, about 3:2:10, about 4:2:10, about 5:2:10, about 6:2:10, about 7:2:10, about 8:2:10, about 9:2:10, about 10:2:10, about 11:2:10, about 12:2:10, about 13:2:10, about 14:2:10, about 15:2:10, about 16:2:10, about 17:2:10, about 18:2:10, about 19:2:10, about 20:2:10, about 1:2:15, about 2:2:15, about 3:2:15, about 4:2:15, about 5:2:15, about 6:2:15, about 7:2:15, about 8:2:15, about 9:2:15, about 10:2:15, about 11:2:15, about 12:2:15, about 13:2:15, about 14:2:15, about 15:2:15, about 16:2:15, about 17:2:15, about 18:2:15, about 19:2:15, about 20:2:15, about 1:2:20, about 2:2:20, about 3:2:20, about 4:2:20, about 5:2:20, about 6:2:20, about 7:2:20, about 8:2:20, about 9:2:20, about 10:2:20, about 11:2:20, about 12:2:20, about 13:2:20, about 14:2:20, about 15:2:20, about 16:2:20, about 17:2:20, about 18:2:20, about 19:2:20, about 20:2:20, about 1:2:25, about 2:2:25, about 3:2:25, about 4:2:25, about 5:2:25, about 6:2:25, about 7:2:25, about 8:2:25, about 9:2:25, about 10:2:25, about 11:2:25, about 12:2:25, about 13:2:25, about 14:2:25, about 15:2:25, about 16:2:25, about 17:2:25, about 18:2:25, about 19:2:25, about 20:2:25, about 1:2:30, about 2:2:30, about 3:2:30, about 4:2:30, about 5:2:30, about 6:2:30, about 7:2:30, about 8:2:30, about 9:2:30, about 10:2:30, about 11:2:30, about 12:2:30, about 13:2:30, about 14:2:30, about 15:2:30, about 16:2:30, about 17:2:30, about 18:2:30, about 19:2:30, about 20:2:30, about 1:2:35, about 2:2:35, about 3:2:35, about 4:2:35, about 5:2:35, about 6:2:35, about 7:2:35, about 8:2:35, about 9:2:35, about 10:2:35, about 11:2:35, about 12:2:35, about 13:2:35, about 14:2:35, about 15:2:35, about 16:2:35, about 17:2:35, about 18:2:35, about 19:2:35, about 20:2:35, about 1:2:40, about 2:2:40, about 3:2:40, about 4:2:40, about 5:2:40, about 6:2:40, about 7:2:40, about 8:2:40, about 9:2:40, about 10:2:40, about 11:2:40, about 12:2:40, about 13:2:40, about 14:2:40, about 15:2:40, about 16:2:40, about 17:2:40, about 18:2:40, about 19:2:40, about 20:2:40, about 1:2:45, about 2:2:45, about 3:2:45, about 4:2:45, about 5:2:45, about 6:2:45, about 7:2:45, about 8:2:45, about 9:2:45, about 10:2:45, about 11:2:45, about 12:2:45, about 13:2:45, about 14:2:45, about 15:2:45, about 16:2:45, about 17:2:45, about 18:2:45, about 19:2:45, about 20:2:45, about 1:2:50, about 2:2:50, about 3:2:50, about 4:2:50, about 5:2:50, about 6:2:50, about 7:2:50, about 8:2:50, about 9:2:50, about 10:2:50, about 11:2:50, about 12:2:50, about 13:2:50, about 14:2:50, about 15:2:50, about 16:2:50, about 17:2:50, about 18:2:50, about 19:2:50, or about 20:2:50, including all ranges and subranges thereof.

In one embodiment, the pre-mixture comprises at least one carboxylic acid chosen from citric acid, at least one cyclodextrin chosen from β-cyclodextrin, and water in a weight ratio ranging from about 0.3:1:0.3 to about 5:1:0.3, and a mole ratio ranging from about 2:1:10 to about 30:1:10, such as about 6:1:10.

Once the particular carboxylic acid(s), cyclodextrin(s), and solvent(s), as well as the desired ratio thereof, are chosen, the pre-mixture may be prepared by any conventional method. As an illustratory example only, the pre-mixture may be prepared by simple mixing of the components at the chosen ratio, preferably with heating. For example, the mixture may be mixed at a temperature of about 40° C. to about 100° C., such as about 50° C. to about 95° C.

After the pre-mixture is prepared, it may then be diluted with additional solvent and other additives to the desired concentration of the final composition according to the disclosure. The additional solvent may be the same as the solvent in the pre-mixture, may be different from the solvent in the pre-mixture, or may be a combination of the solvent in the pre-mixture with one or more solvents other than that in the pre-mixture. In various embodiments, compositions according to the disclosure may contain a ratio of pre-mixture:additional solvent ranging from about 1:99 to about 50:50. For example, compositions according to the disclosure may contain a ratio of pre-mixture:additional solvent ranging from about 2:98 to about 40:60, or about 3:97 to about 35:65, such as about 2.5:97.5, about 5:95, about 7.5:92.5, about 10:90, about 12.5:87.5, about 15:85, about 17.5:81.5, about 20:80, about 25:75, or about 30:70, including all ranges and subranges thereof. In preferred embodiments, the ratio of pre-mixture:additional solvent ranges from about 5:95 to about 15:85.

Thus, in various embodiments, compositions according to the disclosure may have weight ratios of carboxylic acid(s):cyclodextrin(s) ranging from about 1:3 to about 6:1, for example about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, or about 6:1, including all ranges and subranges thereof.

The compositions may have mole ratios of carboxylic acid(s):cyclodextrin(s) ranging from about 0.5:1 to about 25:3, for example about 4:1 to about 15:1, about 5:1 to about 12:1, or about 6:1 to about 10:1, such as, for example, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges thereof.

### B. Methods of Treating the Hair

The disclosure also relates to methods of improving appearance of curly hair by the use of the compositions described herein, for example by improving the curl definition of the hair and/or reducing the volume and/or frizziness of the hair. The methods generally comprise applying any of the compositions according to the disclosure to the hair.

The compositions according to the disclosure may be useful in a variety of settings, either for chemically treated or untreated hair, and/or for natural or unnatural curls. The degree of curliness or curl type of the hair may vary and is not limited. For example, the curls of hair been treated by the compositions disclosed herein may range from slightly wavy to very kinky and coily hair, and may have different textures and colors.

Typically, methods according to the disclosure may include applying an effective amount of a composition disclosed herein to hair having curls, when the hair is dry, wet, damp, or moist when the composition is applied thereto. As used herein, the term “effective amount” refers to an amount sufficient to provide a desired effect to the hair, taking into account the degree of curliness, the length, the volume, and the texture of the hair. In general, from about 0.1 grams to about 5 grams of product is applied per gram of hair, depending on the specific product formulation, hair length, hair volume, and hair style type.

The composition applied to the hair may be distributed evenly by combing through with fingers or a means such as a comb or the like. If desired, after application of the compositions disclosed herein, the hair may be shaped or styled, such as scrunched, twisted, or the like, which may aid in defining the curls. The hair may be then air-dried or dried with a dryer, for example a hood dryer. The composition may be allowed to remain on the hair as a leave-in product for any period of time as desired, for example, about 10, 20, or 30 minutes or longer, such as a few hours or a few days, or until the next washing or rinsing of the hair.

In some embodiments, a composition disclosed herein is used alone on the hair, without the use of other hair treatment compositions. In some other embodiments, the compositions may be applied to the hair before, during, or after another hair treatment or styling composition, such as styling gel, hairspray, etc., is applied to the hair. As such, a composition disclosed herein may be applied to the hair and layered with another composition that is also applied to the hair.

In various embodiments, the methods of treating hair with the compositions according to the disclosure, impart various long-lasting benefits described above to the hair, without a greasy feel or flaking, relative to hair not having been treated with a composition according to the disclosure. The term “long-lasting,” as used herein, means that the curl-defining, volume-reducing, and/or frizz-control effect and other benefits imparted to the hair may remain a desired length of time, such as at least one or a few hours, a few days, or until the hair is rinsed or washed.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the terms “applying a composition onto keratin materials” and “applying a composition onto hair” and variations of these phrases are intended to mean contacting the keratin materials including hair and skin, with at least one of the compositions of the disclosure, in any manner. It may also mean contacting the keratin materials in an effective amount.

Unless otherwise indicated, all percentages herein are by weight, relative to the weight of the total composition.

As used herein, the term “curly hair” refers to any hair including a curl. The curl may be natural or unnatural, i.e., formed by chemical treatment or physical treatment of the hair. The degree of curliness of the hair may vary and is not limited.

As used herein, hair with improved or enhanced curl definition may have curls with a shape that has a clean ringlet appearance rather than being frizzy, curls that appear more individualized, curls that are more closed in appearance, and/or curls that have an improved visual appearance of the hair color and/or highlights.

A “leave-in” composition or product refers to a composition such as a hair-treatment composition that is not rinsed and/or washed away with water or acceptable solvent after the application of the composition onto the keratin fiber, such as hair; instead, the composition is allowed to remain on the keratin fibers for a period of time as desired, such from 1 hour, 2 hours, 3 hours, 4 hours, up to 8 hours, overnight, or as long as needed, until next time of washing or rinsing the keratin fibers.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “salts” refers to throughout the disclosure may include salts having a counterion such as an alkali metal, alkaline earth metal, or ammonium counterion. This list of counterions, however, is non-limiting.

It is to be understood that, with regard to salts of carboxylic acids described herein, it is intended to encompass the use of a salt of a carboxylic acid as an ingredient added to a composition according to the disclosure, or to the salt of the carboxylic acid that forms when the carboxylic acid is used as an ingredient in a composition according to the disclosure.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following Examples are intended to be non-limiting and explanatory in nature only. In the Examples, amounts are expressed in percentage by weight (wt %) of active materials, relative to the total weight of the composition, unless otherwise indicated.

Methods for Analysis of Curl Definition and/or Frizz Control

Hair treated in the Examples below was evaluated visually, and/or was analyzed by the RUMBA (Bossa Nova Technologies, LLC) and/or with photographic images for determining hair volume and area.

The RUMBA system, used to assess curl definition, is a polarization imaging system that takes successive images of a hair sample illuminated with infrared LED and under several polarization states, using a rotating polarizer. The images give a modulated intensity on each pixel, which is caused by the birefringence of the hair fiber and contains the angle signature that can be extracted with the system's software to build an orientation image of the sample.

The orientation data can be mapped onto a 2D image to visualize the whole sample. From this orientation data, a coefficient that relates to straightness of the hair can be defined—the straighter the hair fibers in the swatch, the closer to a single angle value the distribution of the orientation will be. The standard deviation of such a distribution is therefore inversely proportional to the perception of straightness.

The same process is done for the alignment, where the alignment of a hair swatch is defined as the hair fibers being “locally straight.” Thus, a straightness coefficient is defined on a small scale of the 2D mapping of the orientation, which allows for a straightness coefficient to be obtained on every pixel of the image.

### Example 1A

### Compositions

A pre-mixture according to the disclosure was prepared as set forth in Table 1-1.

TABLE 1-1
Pre-Mixture

| COMPONENT | AMOUNT (g) |
|---|---|
| CITRIC ACID | 1.15 |
| β-CYCLODEXTRIN | 1.32 |
| WATER | 0.18 |

The components in Table 1-1 were mixed at a temperature of about 85° C. for a period of about 2-3 hours, to prepare a pre-mixture according to the disclosure.

An inventive composition containing 5% of the pre-mixture of Table 1-1 was prepared by diluting it with additional water (premixture:additional water=5:95), and four comparative compositions were prepared as set forth in Table 1-2. The admixture of composition C4 was prepared by mixing 100 mL of a solution of 4.4% citric acid in water with 100 mL of a solution of 4.2% β-cyclodextrin solution in water.

TABLE 1-2
Compositions

| | Inventive | Comparative | | | |
|---|---|---|---|---|---|
| COMPONENT | 1A | C1 | C2 | C3 | C4 |
| PRE-MIXTURE | 5 | — | — | — | — |
| CITRIC ACID | — | — | 2.2 | — | — |
| β-CYCLODEXTRIN | — | — | — | 2.1 | — |
| ADMIXTURE | — | — | — | — | 4.3 |
| WATER | QS to | QS to | QS to | QS to | QS to |
| | 100 | 100 | 100 | 100 | 100 |

Each of compositions 1A and C1-C4 was adjusted to a pH of about 4 with a 0.1 M solution of sodium hydroxide.

### Example 1B

### Demonstration of Effects on Hair

In order to demonstrate the beneficial effects of compositions according to the disclosure on curly hair, approximately 0.4 grams of each of compositions 1A and C1-C4 was applied to three separate swatches of curly hair (Caucasian). The compositions were massaged through the hair to ensure even distribution, and the treated hair was scrunched five times by hand. The swatches were then left to dry overnight in a chamber having a relative humidity of 20%. After overnight drying, the swatches were evaluated.

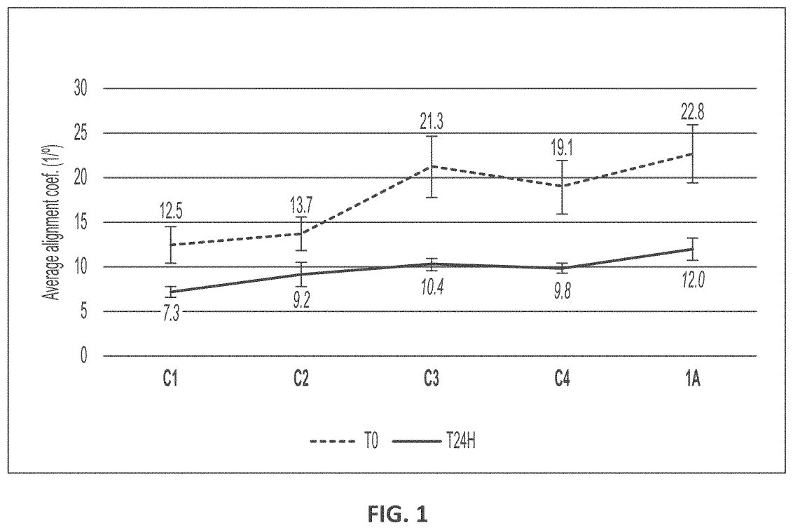

Using the RUMBA system, the treated hair was first analyzed after drying overnight (T0), again after combing (T0_{comb}), and again after 24 hours of exposure to conditions of 80% relative humidity (T24H). Based on analysis by the RUMBA system, as can be seen in FIG. 1, the swatches treated with composition C1 led to hair with the least curl definition, followed by composition C2, C4, and C3, respectively, whereas composition 1A according to the disclosure had the most defined curls, at both TO and T24H. The following Table 1-3 summarizes the results of this analysis.

TABLE 1-3
Average Alignment Coefficient (1/°)

| | T0 | T24 H |
|---|---|---|
| C1 | 7.3 | 12.5 |
| C2 | 9.2 | 13.7 |
| C3 | 10.4 | 21.3 |
| C4 | 9.8 | 19.1 |
| 1A | 12.0 | 22.8 |

FIG. 1 is a graphical representation of the data in Table 1-3, with the bars at each point representing the standard deviation.

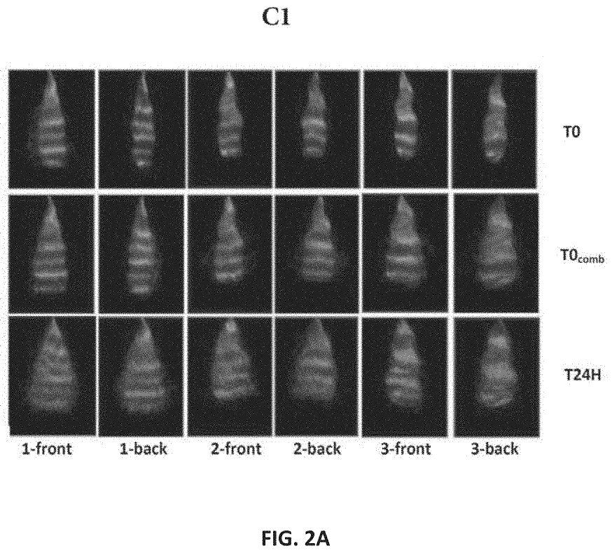

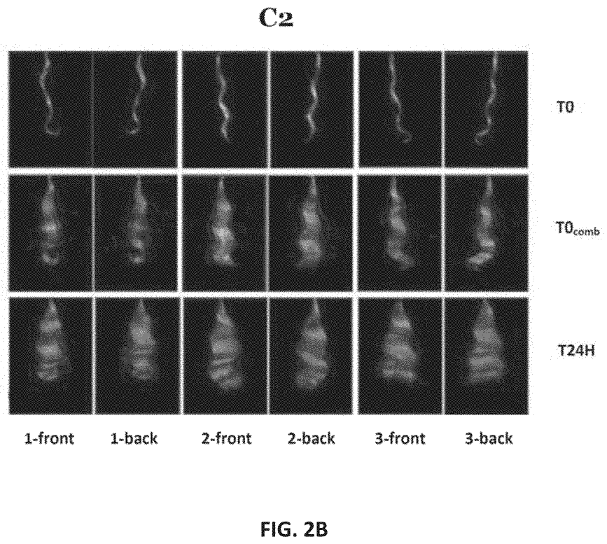

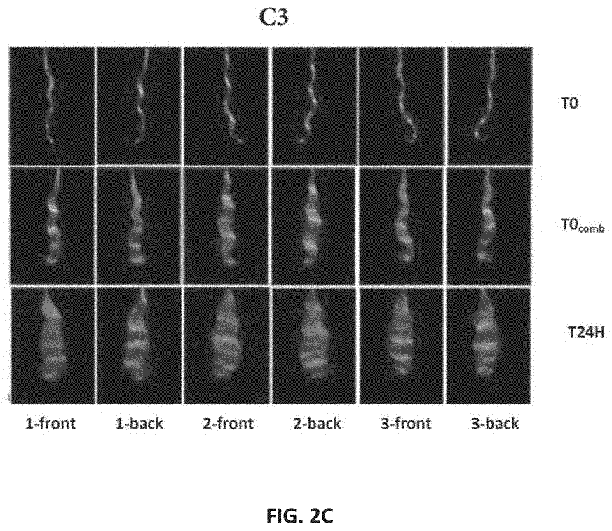

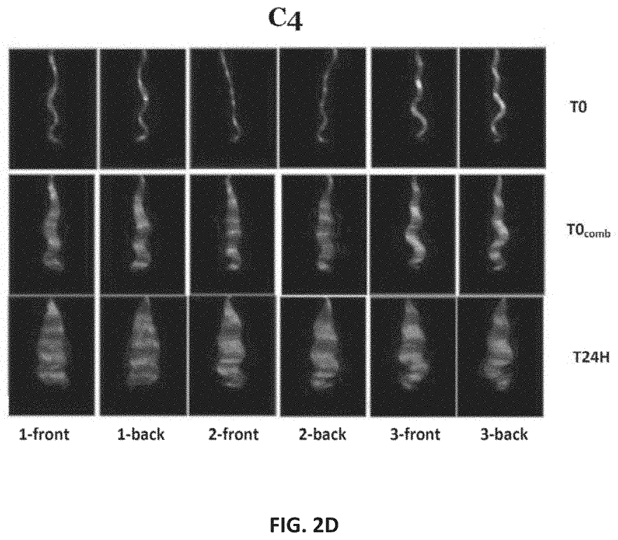

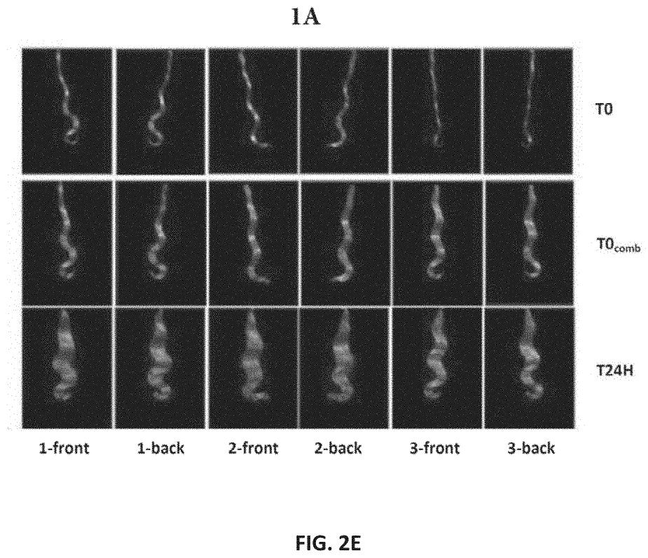

FIGS. 2A-2E show images of the hair swatches that were treated with comparative compositions C1-C4 and inventive composition 1A, obtained with the RUMBA analyzer. Each figure shows the front and back image of each of the three swatches treated with each composition, at T0, T0_{comb}, and T24H. FIGS. 2A-2E confirm visually what is shown in Table 1-3 and FIG. 1, i.e. that the hair treated with composition 1A according to the disclosure demonstrated better curl definition and frizz control than the hair treated with the comparative compositions C1-C4, particularly noticeable after the hair was subjected to high humidity for 24 hours (T24H).

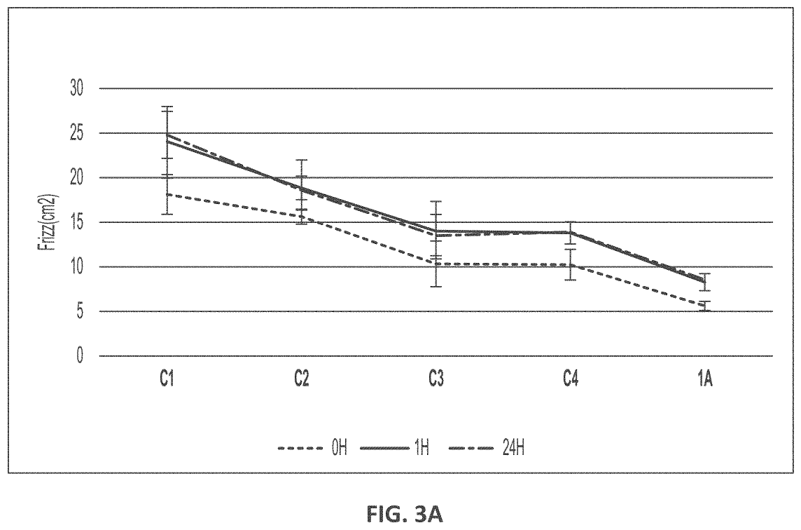

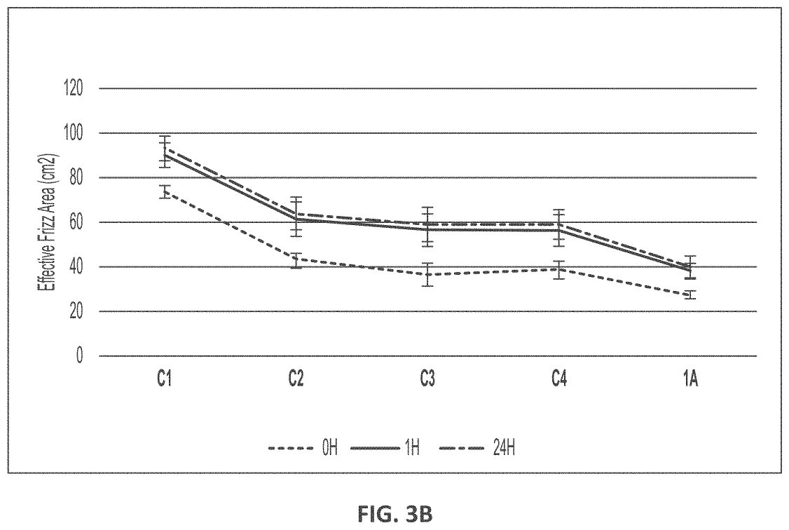

The treated hair swatches were also analyzed under environmentally controlled conditions (temperature and humidity), wherein photographic images of the swatches were obtained at specified times and assessed for changes in volume and area of the hair over time, in order to evaluate frizz. The treated hair was first analyzed after drying overnight (T0), and after 0.5 hours (T0.5H), 1 hour (T1H), 2 hours (T2H), and 24 hours (T24H) of exposure to conditions of 80% relative humidity, to measure the frizz and frizz area of each swatch. As can be seen in FIGS. 3A-3B, the swatches treated with composition C1 led to hair with the least frizz control, followed by composition C2, C3, and C4 respectively, whereas composition 1A according to the disclosure provided the most frizz control.

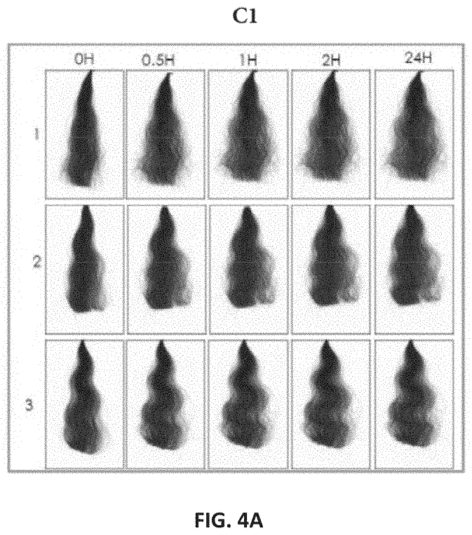

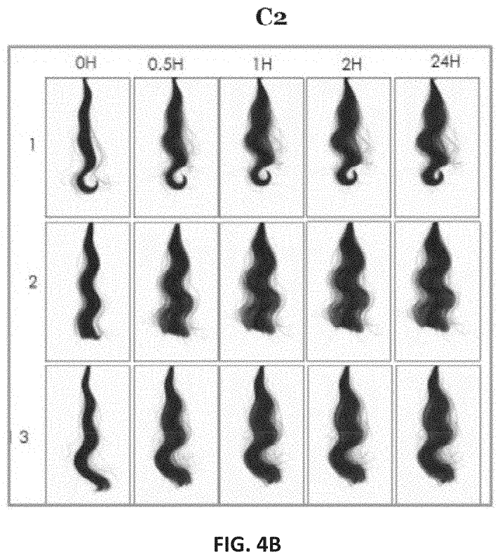

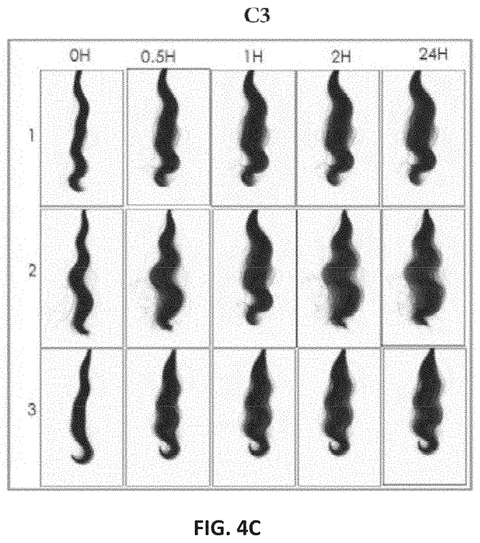

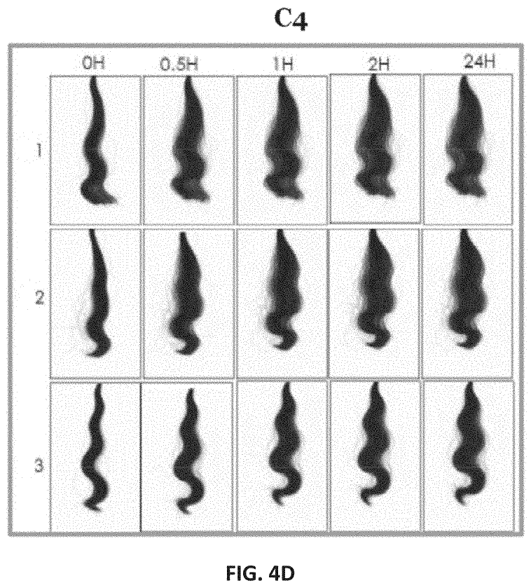

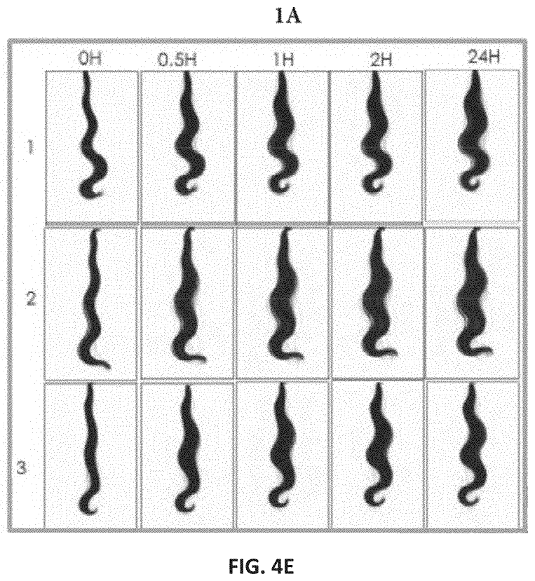

FIGS. 4A-4E show images of the hair swatches that were treated with comparative compositions C1-C4 and inventive composition 1A. Each figure shows the three swatches treated with each composition, at T0, T0.5H, T1H, T2H, and T24H. FIGS. 4A-4E confirm visually that the hair treated with composition 1A according to the disclosure demonstrated better frizz control than the hair treated with the comparative compositions C1-C4, which is particularly noticeable after the hair was subjected to high humidity for 24 hours (T24H).

Example 2A

### Demonstration of Benefit Across Rance of Mole Ratios

To demonstrate improved frizz control of hair treated with compositions according to the disclosure over a range of mole ratios of carboxylic acid:cyclodextrin, two compositions were prepared according to the disclosure, containing mole ratios of 6:1 (Composition 2A) and 10:1 (Composition 2B) citric acid:β-cyclodextrin in water, following the same procedure as in Example 1A, and compared to composition C1 from Example 1A.

Approximately 0.4 grams of each of compositions 2A, 2B, and C1 was applied to three separate swatches of curly hair (Caucasian). The compositions were massaged through the hair to ensure even distribution, and the treated hair was scrunched five times by hand. The swatches were left to air dry overnight. After overnight drying, the swatches were evaluated.

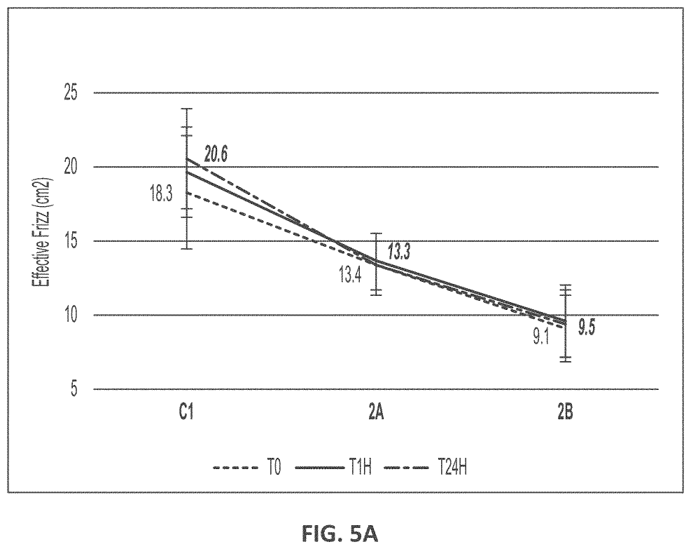

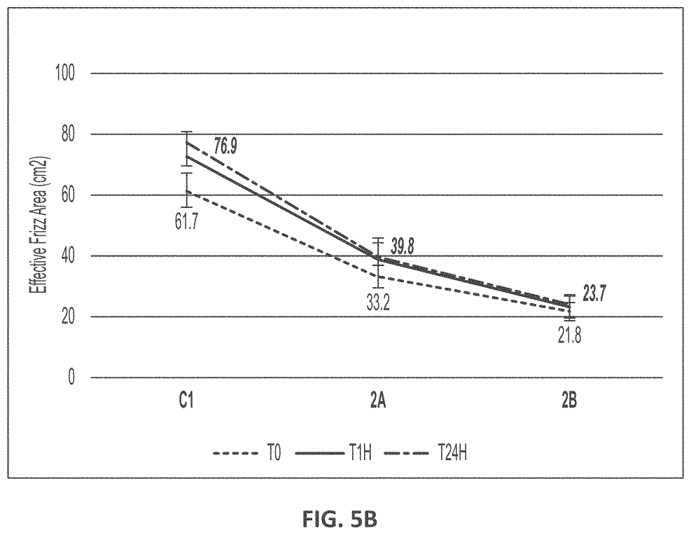

The treated hair swatches were also analyzed under environmentally controlled conditions (temperature and humidity), wherein photographic images of the swatches were obtained at specified times and assessed for changes in volume and area of the hair over time, in order to evaluate frizz. The treated hair was first analyzed after drying overnight (T0), and after 1 hour (T1H) and 24 hours (T24H) of exposure to conditions of 80% relative humidity, to measure the frizz and frizz area of each swatch. As can be seen in FIGS. 5A-5B, the swatches treated with composition C1 led to hair with the least frizz control, whereas composition 2A and 2B according to the disclosure provided frizz control to the treated hair, with the hair treated with composition 2B demonstrating the greatest degree of frizz control.

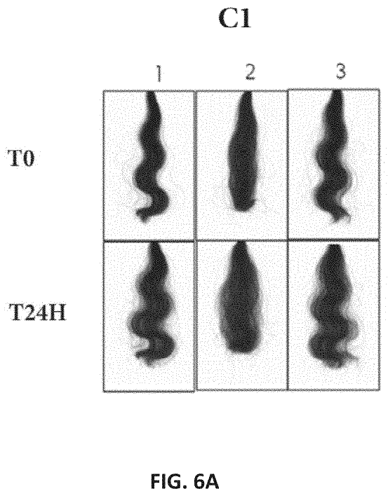

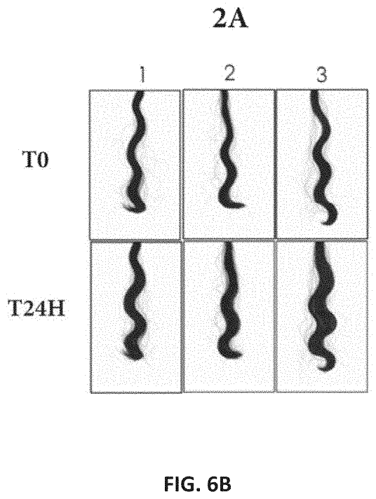

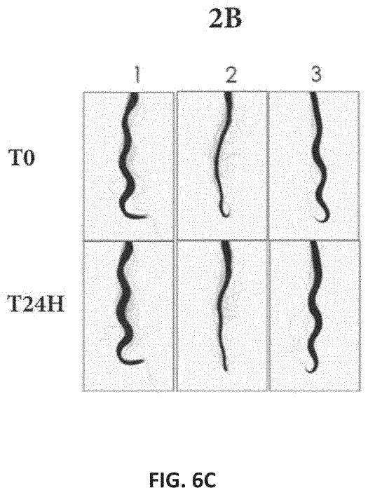

FIGS. 6A-6C show images of the hair swatches that were treated with comparative composition C1 and inventive compositions 2A and 2B. Each figure shows the three swatches treated with each composition, at T0 and T24H. FIGS. 6A-6C confirm visually that the hair treated with compositions 2A and 2B according to the disclosure demonstrated better frizz control than the hair treated with the comparative composition C1, at both T0 and T24H.

Example 2B

### Demonstration of Benefit Across Rance of Mole Ratios

To demonstrate improved curl definition and frizz control of hair treated with compositions according to the disclosure across a range of mole ratios, two pre-mixtures were prepared according to the disclosure following the procedure in Example 1A to prepare pre-mixtures having mole ratios of 6:1:10 (pre-mixture 2C) and 3:1:10 (pre-mixture 2D) citric acid:B-cyclodextrin:water, following the same procedure as in Example 1A. Pre-mixtures 2C and 2D were then mixed with water at a ratio of 5:95 pre-mixture:water to prepare Compositions 2C and 2D, which were compared to composition C1 from Example 1A.

Approximately 0.4 grams of each of compositions 2C, 2D, and C1 was applied to two separate swatches of curly hair (Caucasian). The compositions were massaged through the hair to ensure even distribution, and the treated hair was scrunched five times by hand. The swatches were left to air dry overnight.

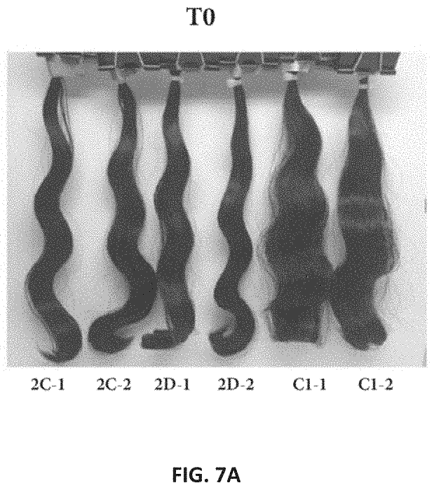

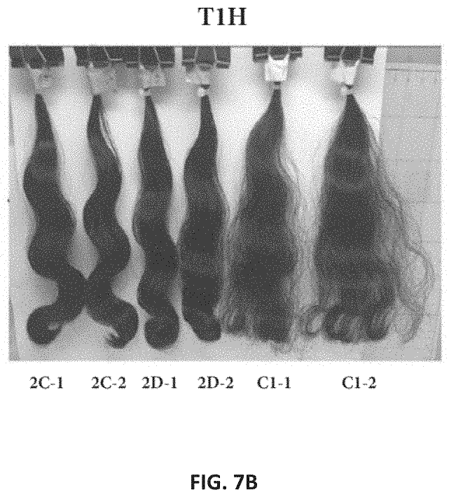

After overnight drying, the swatches were evaluated visually (T0), and then subjected to conditions of 80% relative humidity and evaluated visually again after 1 hour (T1H), shown in FIGS. 7A-7B. As seen in the figures, after an hour at high humidity, the hair swatches treated with both Compositions 2C and 2D have significantly greater curl definition and frizz control than the swatches treated with composition C1.

### Example 2C

### Demonstration of Benefit Across Rance of Mole Ratios

To demonstrate improved curl definition and frizz control of hair treated with compositions according to the disclosure across a range of mole ratios, four pre-mixtures were prepared according to the disclosure following the procedure in Example 1A to prepare pre-mixtures having mole ratios of 2:1:10 (pre-mixture 2E), 6:1:10 (pre-mixture 2F), 15:1:10 (pre-mixture 2G), and 20:1:10 (pre-mixture 2H) citric acid:B-cyclodextrin:water, following the same procedure as in Example 1A. The pre-mixtures were then mixed with water at a ratio of 5:95 pre-mixture:water, to prepare Compositions 2E-2H.

Approximately either 0.15 grams (A) or 0.4 grams (B) of each of compositions 2E-2H was applied to two separate swatches of curly hair (Caucasian). The compositions were massaged through the hair to ensure even distribution, and the treated hair was scrunched five times by hand. The swatches were left to air dry overnight.

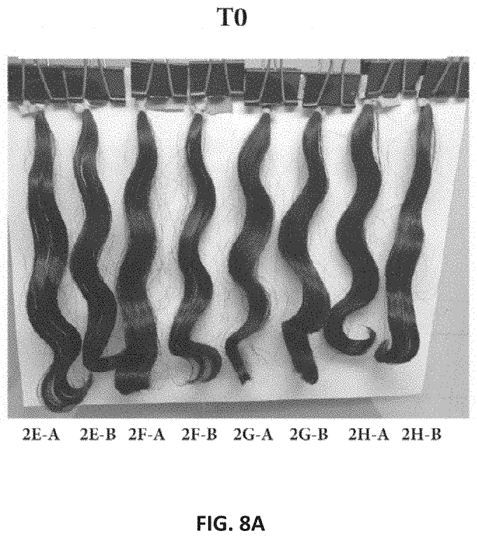

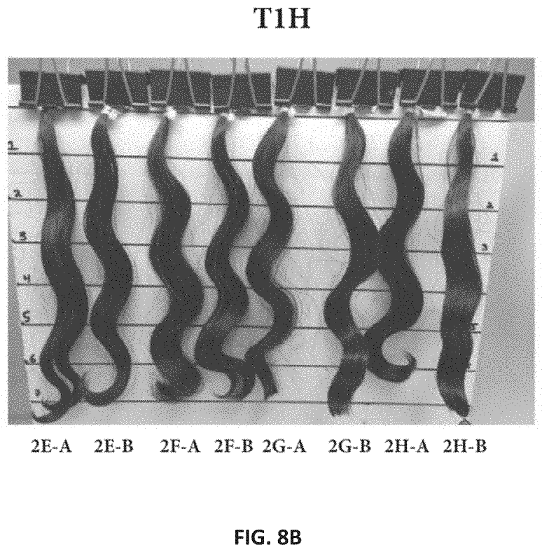

After overnight drying, the swatches were evaluated visually (T0), and then subjected to conditions of 80% relative humidity and evaluated visually again after 1 hour (T1H), shown in FIGS. 8A-8B. As seen in the figures, after an hour at high humidity, the hair swatches demonstrate that curl definition, frizz control, or both is maintained across a range of mole ratios.

### Example 3

### Demonstration of Benefit Across Rance of Concentrations

To demonstrate improved curl definition and frizz control of hair treated with compositions according to the disclosure across a range of concentrations of pre-mixture in the final composition, four compositions were prepared. A pre-mixture having a mole concentration of 6:1:10 citric acid:β-cyclodextrin:water was prepared as in Example 1A, and was then mixed with additional water at ratios of pre-mixture:water of 2.5:97.5 (Composition 3A), 5:95 (Composition 3B), 15:85 (Composition 3C), and 30:70 (Composition 3D).

Approximately either 0.15 grams (A) or 0.4 grams (B) of each of compositions 3A-3D was applied to two separate swatches of curly hair (Caucasian). The compositions were massaged through the hair to ensure even distribution, and the treated hair was scrunched five times by hand. The swatches were left to air dry overnight.

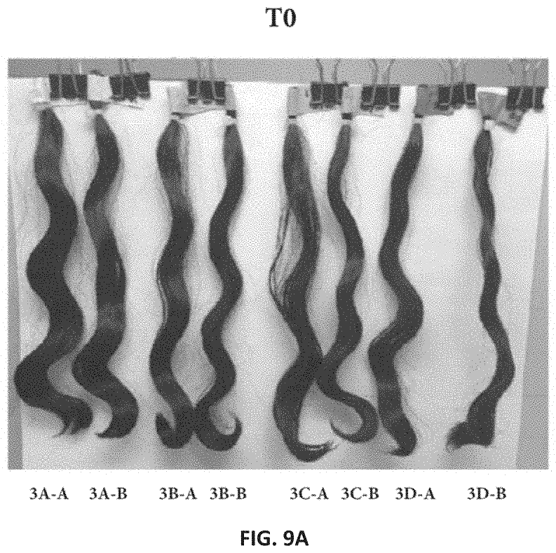

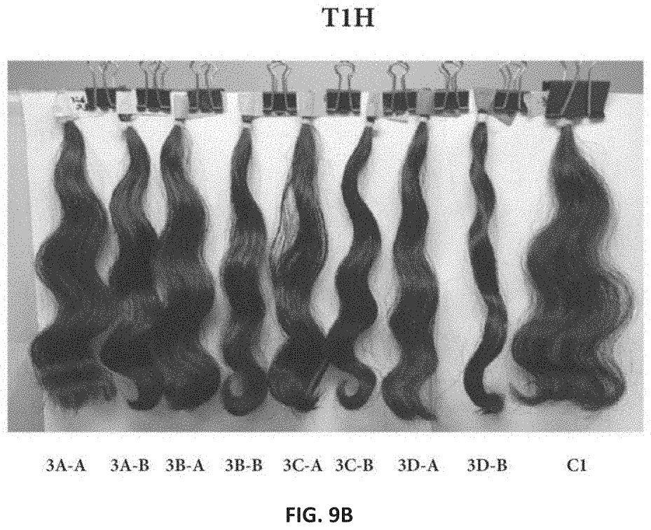

After overnight drying, the swatches were evaluated visually (TO), and then subjected to conditions of 80% relative humidity and evaluated visually again after 1 hour (T1H), shown in FIGS. 9A-9B. The swatches after 1 hour were compared against a swatch treated with Composition C1 from Example 1A. As seen in the figures, after an hour at high humidity, the hair swatches treated with compositions 3A-3D, at both concentrations of composition applied to hair, show that curl definition, frizz control, or both is maintained across a range of concentrations of pre-mixture in the final composition.

### Example 4A

### Compositions With Rance of Carboxylic Acids

To demonstrate that the benefits of improved curl definition and/or frizz control are imparted to curly hair using other carboxylic acids according to the disclosure, six compositions were prepared with the following amounts of acid, β-cyclodextrin, and water.

TABLE 4-1

| Composition | | Acid (%) | β-cyclodextrin (%) | Water (%) |
|---|---|---|---|---|
| 4A | Lactic acid* | 1.5 | 1.5 | QS to 100 |
| 4B | Malic acid | 1.4 | 1.5 | QS to 100 |
| 4C | Maleic acid | 1.1 | 1.1 | QS to 100 |
| 4D | Tartaric acid | 0.8 | 1.5 | QS to 100 |
| 4E | Glycolic acid | 1.5 | 1.5 | QS to 100 |
| 4F | Phytic acid** | 1.2 | 1.1 | QS to 100 |

*added as 90% active material in water
**added as 50% active material in water

Each of compositions 4A-4F was adjusted to a pH of about 3.5-4.0 with a 0.1 M solution of sodium hydroxide.

### Example 4B

### Demonstration of Hair Benefit With Rance of Carboxylic Acids

To demonstrate improved curl definition and frizz control of hair treated with compositions according to the disclosure across a range of carboxylic acids, three separate swatches of curly hair (Caucasian) were washed with a standard shampoo, rinsed, and excess water was removed by hand. Subsequently, approximately 0.4 grams of each of compositions 4A-4F or C1 from Example 1A was applied to each of the three swatches of hair.

The compositions were massaged through the hair to ensure even distribution, and the treated hair was scrunched five times by hand. The swatches were then left to dry overnight in a chamber having a relative humidity of 20%. After overnight drying, the swatches were evaluated.

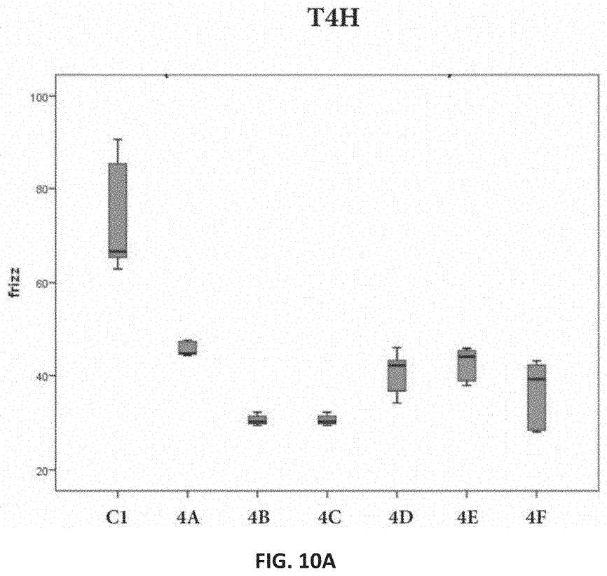

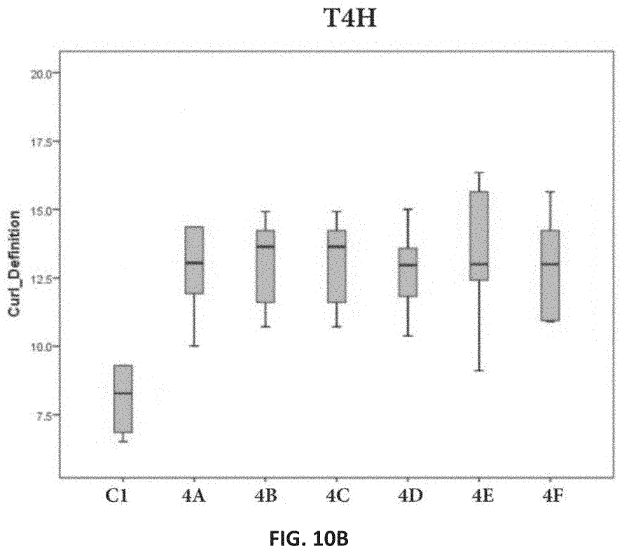

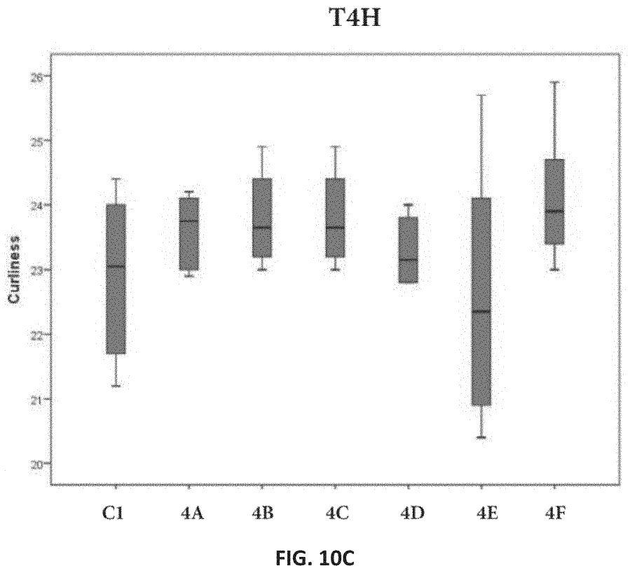

Using the RUMBA system, images of the treated hair were obtained after drying overnight (T0), and again after 4 hours of exposure to conditions of 80% relative humidity (T4H). Based on analysis by the RUMBA system, as can be seen in FIGS. 10A-10C, the swatches treated with composition C1 showed the least frizz control and curl definition, whereas each of compositions 4A-4F provided improved frizz control and/or curl definition to the hair.

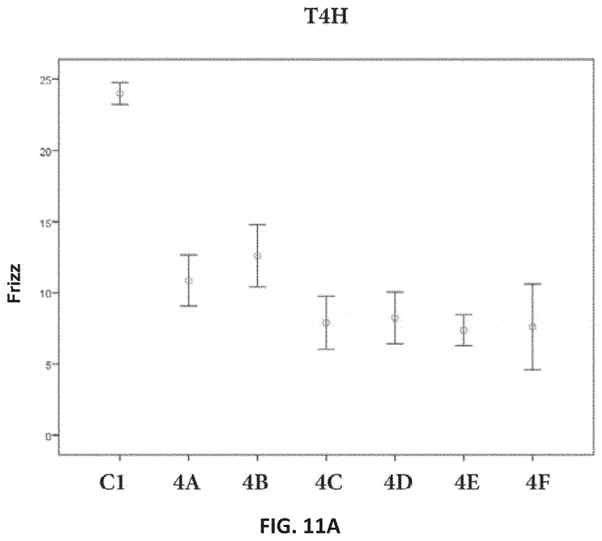

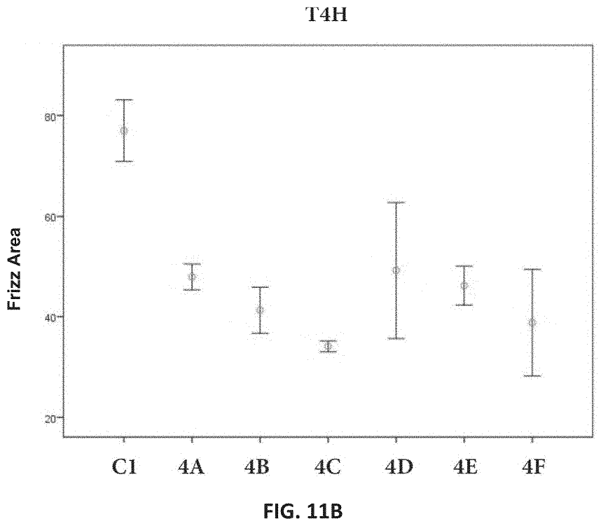

The treated hair swatches were also analyzed under environmentally controlled conditions (temperature and humidity), wherein photographic images of the swatches were obtained at specified times and assessed for changes in volume and area of the hair over time, in order to evaluate frizz. The treated hair was analyzed after 4 hours (T4H) of exposure to conditions of 80% relative humidity. As can be seen in FIGS. 11A-11B, the swatches treated with composition C1 led to hair with the least frizz control, whereas compositions 4A-4F according to the disclosure provided frizz control to the treated hair, even for those compositions having higher water content.

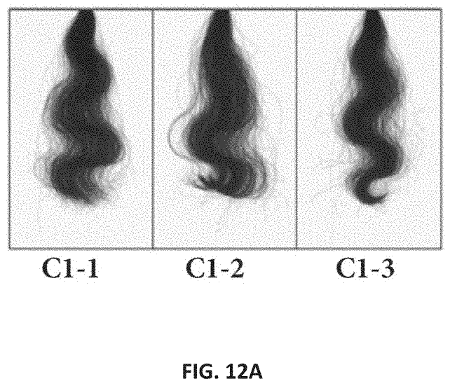

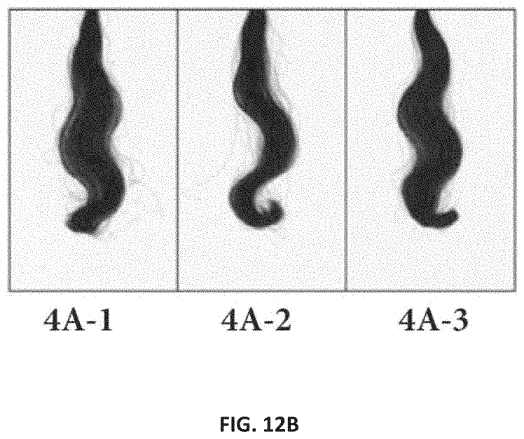

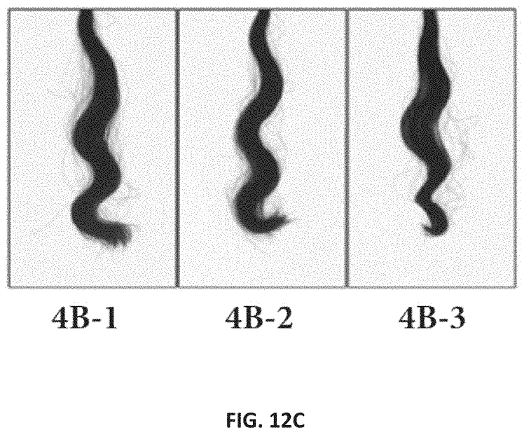

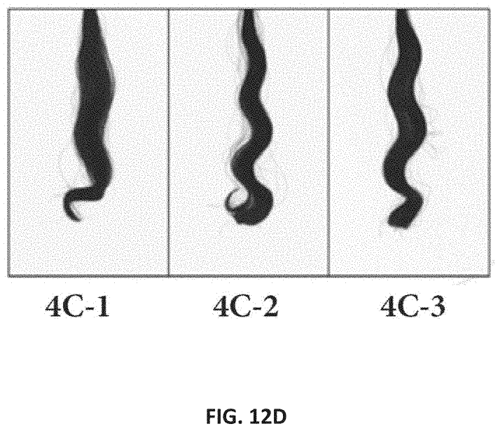

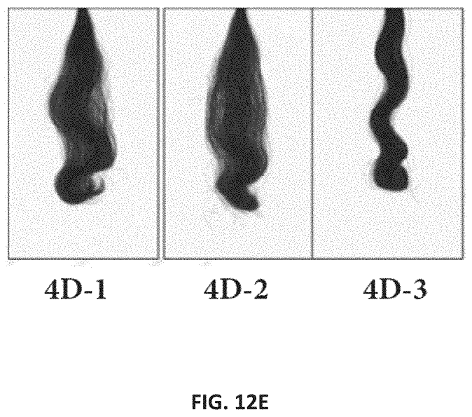

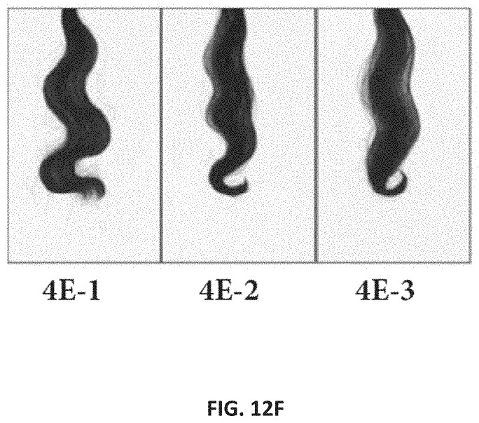

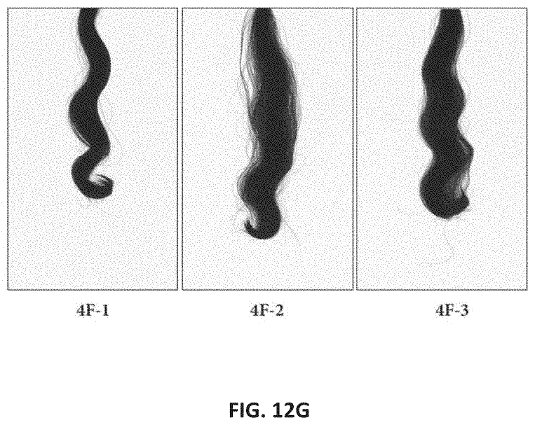

FIGS. 12A-12G show images of the hair swatches that were treated with comparative composition C1 and inventive compositions 4A-4F at T4H. FIGS. 12A-12G confirm visually that the hair treated with compositions 4A-4F according to the disclosure demonstrated better frizz control than the hair treated with the comparative composition C1.

The above Examples demonstrate that compositions according to the disclosure provide improved curl definition and/or frizz control to hair, which lasts over time even after exposure to high humidity, over a range of different mole ratios, concentrations, and components.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-64. (canceled)

65. A composition for treating keratin fibers, comprising:
(a) at least one carboxylic acid or salt thereof;
(b) at least one cyclodextrin or derivative thereof; and
(c) at least one solvent;
wherein the composition has a mole ratio of (a):(b) ranging from about 0.5:1 to about 25:3 and/or a weight ratio of (a):(b) ranging from about 1:3 to about 6:1.

66. The composition according to claim 65, wherein the mole ratio of (a):(b) ranges from about 4:1 to about 15:1.

67. The composition according to claim 65, wherein the weight ratio of (a):(b) ranges from about 1:3 to about 6:1.

68. The composition according to claim 65, comprising at least one carboxylic acid or salt thereof chosen from mono-carboxylic acids, di-carboxylic acids, tri-carboxylic acids, salts thereof, or mixtures thereof.

69. The composition according to claim 65, comprising at least one carboxylic acid or salt thereof chosen from citric acid, lactic acid, malic acid, maleic acid, tartaric acid, glycolic acid, phytic acid, salts thereof, or mixtures thereof.

70. The composition according to claim 65, wherein the total amount of carboxylic acids and salts thereof ranges from about 0.01% to about 20% by weight, relative to the total weight of the composition.

71. The composition according to claim 65, wherein the at least one cyclodextrin or derivative thereof is chosen from those of formula: 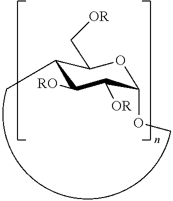
wherein:
R is chosen from H, CH₃, or a hydroxypropyl group, and n ranges from 6-8.

72. The composition according to claim 65, wherein the at least one cyclodextrin or derivative thereof is chosen from α-cyclodextrin, β-cyclodextrin, γ-cyclodextrin, methyl derivatives of α-cyclodextrin, β-cyclodextrin, or γ-cyclodextrin, hydroxypropyl derivatives of α-cyclodextrin, β-cyclodextrin, or γ-cyclodextrin, or mixtures thereof.

73. The composition according to claim 65, wherein the at least one cyclodextrin or derivative thereof is present in an amount ranging from about 0.01% to about 20% by weight, relative to the total weight of the composition.

74. The composition according to claim 65, wherein the solvent comprises water and optionally at least one additional solvent chosen from ethylene glycol, propylene glycol, butylene glycol, hexylene glycol, propanediol, glycerin, ethyl alcohol, isopropyl alcohol, propyl alcohol, benzyl alcohol, phenylethyl alcohol, glycols or glycol ethers such as monomethyl, monoethyl and monobutyl ethers of ethylene glycol, propylene glycol butylene glycol, hexylene glycol, or dipropylene glycol, alkyl ethers of diethylene glycol such as monoethyl ether or monobutyl ether of diethylene glycol, or mixtures thereof.

75. The composition according to claim 65, comprising:
(a) at least one carboxylic acid having a molecular weight of less than about 900 g/mol, salts thereof, or mixtures thereof;
(b) at least one cyclodextrin or derivative thereof chosen from those of formula: 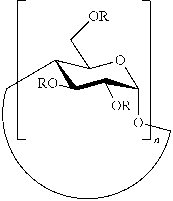
wherein:
R is chosen from H, CH₃, or a hydroxypropyl group, and n ranges from 6-8; and
(c) water and optionally at least one additional solvent;
wherein the composition has a mole ratio of (a):(b) ranging from about 4:1 to about 15:1.

76. The composition according to claim 65, comprising:
(a) at least one carboxylic acid chosen from citric acid, lactic acid, malic acid, maleic acid, tartaric acid, glycolic acid, phytic acid, salts thereof, or mixtures thereof;
(b) at least one cyclodextrin or derivative thereof chosen from α-cyclodextrin, β-cyclodextrin, γ-cyclodextrin, methyl derivatives thereof, hydroxypropyl derivatives thereof, or mixtures thereof; and
(c) water and optionally at least one additional solvent;
wherein the composition has a mole ratio of (a):(b) ranging from about 4:1 to about 15:1.

77. The composition according to claim 65, wherein the pH of the composition ranges from about 2 to about 6.

78. A composition comprising:
(a) from about 0.1% to about 10% of at least one carboxylic acid chosen from citric acid, lactic acid, malic acid, maleic acid, tartaric acid, glycolic acid, phytic acid, salts thereof, or mixtures thereof;
(b) from about 0.1% to about 10% of at least one cyclodextrin chosen from α-cyclodextrin, β-cyclodextrin, γ-cyclodextrin, methyl derivatives thereof, hydroxypropyl derivatives thereof, or mixtures thereof; and
(c) water and optionally at least one additional solvent;
wherein the composition has a mole ratio of (a):(b) ranging from about 5:1 to about 12:1 and/or a weight ratio of (a):(b) ranging from about 1:3 to about 6:1; and
wherein all amounts are by weight, relative to the total weight of the composition.

79. The composition according to claim 78, wherein the at least one carboxylic acid or salt thereof is chosen from citric acid, lactic acid, malic acid, maleic acid, tartaric acid, glycolic acid, phytic acid, salts thereof, or mixtures thereof.

80. The composition according to claim 78, wherein the at least one cyclodextrin or derivative thereof is chosen from α-cyclodextrin, β-cyclodextrin, γ-cyclodextrin, methyl derivatives of α-cyclodextrin, β-cyclodextrin, or γ-cyclodextrin, hydroxypropyl derivatives of α-cyclodextrin, β-cyclodextrin, or γ-cyclodextrin, or mixtures thereof.

81. The composition according to claim 78, wherein the pH of the composition ranges from about 2 to about 6.

82. A method of treating hair comprising applying to the hair a composition comprising:
(a) at least one carboxylic acid or salt thereof;
(b) at least one cyclodextrin or derivative thereof; and
(c) at least one solvent;
wherein the composition has a mole ratio of (a):(b) ranging from about 0.5:1 to about 25:3 and/or a weight ratio of (a):(b) ranging from about 1:3 to about 6:1.

83. The method according to claim 82, which is a method of improving the definition of hair curls.

84. The method according to claim 82, which is a method of providing frizz and/or volume control to the hair.
